(19) United States
(12) Patent Application Publication
(10) Pub. No.: US 2026/0294487 A1
(43) Pub. Date: Oct. 1, 2026

(54) ROD-TO-ROD CONNECTOR ASSEMBLY FOR SPINAL IMPLANT

(71) Applicant: Warsaw Orthopedic, Inc., Warsaw, IN (US)

(72) Inventors: William Alan Rezach, Covington, TN (US); Carrie J. Breuer, Memphis, TN (US)

(21) Appl. No.: 19/478,856

(22) PCT Filed: Apr. 29, 2024
(86) PCT No.: PCT/IB2024/054150

§ 371 Date: Oct. 27, 2025

Related U.S. Application Data
(60) Provisional application No. 63/498,668, filed on Apr. 27, 2023.
(60) Provisional application No. 63/552,249, filed on Feb. 12, 2024.

(51) Int. Cl. A61B 17/70; A61B 17/00

(52) U.S. Cl. CPC A61B 17/7052; A61B 2017/00526

(57) ABSTRACT

A connector includes a first connector body with a first opening extending therethrough and a support recess extending therethrough. A second connector body has a post extending laterally therefrom. The post is supported by the support recess and has a groove. A fastener is secured within the first connector body and has an end that is positioned within the support recess and the groove. A crown is supported by the first connector body and is positioned within the first opening. The crown includes a first crown recess that engages the post and a second crown recess that is opposite the first crown recess. The second connector body is translatable and rotatable relative to the first connector body to adjust a position of the first opening relative to the second opening.

### CROSS-REFERENCE TO RELATED APPLICATIONS

This application claims the benefit of and priority to U.S. Provisional Patent Application No. 63/552,249 filed Feb. 12, 2024 and U.S. Provisional Patent Application No. 63/498,668 filed Apr. 27, 2023. The entire disclosure of the above applications is incorporated herein by reference.

### TECHNICAL FIELD

The present disclosure relates to a domino connector (e.g., a rod-to-rod connector) for connecting a first spinal rod to a second spinal rod during a spinal procedure.

### BACKGROUND

Treatment of spinal disorders, such as degenerative disc disease, disc herniations, scoliosis or other curvature abnormalities, and fractures, often requires surgical treatments. For example, implants may be used to preserve motion between vertebral members.

Surgical treatment typically involves the use of longitudinal members, such as spinal rods. Spinal rods may be attached to the exterior of two or more vertebral members to assist with the treatment of a spinal disorder. Spinal rods may provide a stable, rigid column that helps bones to fuse, and may redirect stresses over a wider area away from a damaged or defective region. Also, rigid spinal rods may help in spinal alignment. During surgical treatment, one or more spinal rods and bone fasteners can be delivered to a surgical site. The rods may be attached via the fasteners to the exterior of two or more vertebral members. In complex spinal procedures, a first spinal rod may need to be connected to a second spinal rod. For example, during cervical thoracic (CT) procedures, a first spinal rod that is coupled to the cervical portion of the spine may need to be coupled to a second spinal rod that is coupled to the thoracic portion of the spine.

### SUMMARY

According to one embodiment, a connector for use in a spinal procedure is disclosed. The connector includes a first connector body that has a first opening extending therethrough along a first axis and a support recess extending therethrough along a second axis that is transverse to the first axis. The connector further includes a second connector body that has a second opening extending therethrough and a post extending laterally therefrom. The post is supported by the support recess and has a groove. A fastener is secured within the first connector body and has an end that is positioned within the support recess and the groove. A crown is supported by the first connector body and is positioned within the first opening. The crown includes a first crown recess that engages the post and a second crown recess that is opposite the first crown recess. The second connector body is translatable and rotatable relative to the first connector body to adjust a position of the first opening relative to the second opening. Surfaces of the support recess and the first crown recess create an interference fit against the post to temporarily maintain the position of the first opening relative to the second opening.

According to another embodiment, a rod-to-rod connector for use in a spinal procedure is disclosed. The rod-to-rod connector includes a first connector body that has a first opening extending therethrough along a first axis and a support recess extending therethrough along a second axis that is transverse to the first axis. The rod-to-rod connector includes a second connector body that has a second opening extending therethrough and a post extending laterally therefrom. The post is supported by the support recess and has a groove. A pin is secured within the first connector body and has a first end and a second end opposite the first end. The second end is positioned within the support recess and the groove. A crown is supported by the first connector body and is positioned within the first opening. The crown includes a first crown recess that engages the post and a second crown recess that is opposite the first crown recess. The second connector body is movable along the second axis to allow adjustment of a distance between the first connector body and the second connector body. The second connector body is movable about the second axis to allow adjustment of an angular position between the first connector body and the second connector body.

According to another embodiment, a method of assembling a rod-to-rod connector for use in a spinal procedure is disclosed. The connector includes a first connector body, a second connector body, a crown, and a fastener. The first connector body has a first axis and a support recess extending along a second axis. The second connector body has a post extending laterally therefrom. The post defines a groove. The crown is positioned within the first connector body and defines a first crown recess. The method includes positioning the crown within the first connector body such that the first crown recess faces the support recess. The method also includes positioning the post of the second connector body between the support recess and the first crown recess such that the groove is positioned within the support recess. The method also includes securing the fastener within the first connector body such that an end of the fastener extends into the recess of the first connector body and the groove of the post. The second connector body being translatable and rotatable relative to the first connector body.

According to another embodiment, a connector for use in a spinal procedure is disclosed. The connector includes a first connector body including a first opening extending therethrough along a first axis and a support recess extending therethrough along a second axis that is transverse to the first axis. The connector also has a second connector body including a post extending laterally therefrom. The post is supported by the support recess and has a groove. A first fastener is secured within the first connector body and has an end that is positioned within the support recess and the groove. A second fastener is secured to and extends from the second connector body. The second fastener is configured to couple to a vertebral member. A crown is supported by the first connector body and is positioned within the first opening. The crown includes a first crown recess that engages the post and a second crown recess that is opposite the first crown recess. The second connector body is translatable and rotatable relative to the first connector body to adjust a position of the first opening relative to the second fastener. Surfaces of the support recess and the first crown recess create an interference fit against the post to temporarily maintain the position of the first opening relative to the second fastener.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1A illustrates a perspective view of a rod-to-rod connector according to one embodiment and including a first connector body, a first spinal rod, a second connector body, and a second spinal rod, according to some aspects of the present disclosure.

FIG. 1B illustrates another perspective view of the rod-to-rod connector of FIG. 1A with the first spinal rod and the second spinal rod removed.

FIG. 2A illustrates an exploded view of the connector of FIG. 1B.

FIG. 2B illustrates an exploded view of a portion of the connector of FIG. 1B.

FIG. 3 illustrates a side view of the second connector body of FIG. 1B.

FIG. 4 illustrates an end view of the second connector body of FIG. 1B.

FIG. 5 illustrates a cross-sectional view of the connector of FIG. 1B along the line 5—5 of FIG. 1B.

FIG. 6 illustrates a side view of the connector of FIG. 1B in a first axial position.

FIG. 7 illustrates a cross-sectional view of the connector in the first axial position and taken along the line 7—7 of FIG. 1B.

FIG. 8 illustrates a side view of the connector of FIG. 1B in a second axial position.

FIG. 9 illustrates a cross-sectional view of the connector in the second axial position and taken along the line 9—9 of FIG. 1B.

FIG. 10 illustrates a side view of the connector of FIG. 1B in a third axial position.

FIG. 11 illustrates a cross-sectional view of the connector in the third axial position and taken along the line 11—11 of FIG. 1B.

FIG. 12 illustrates a side view of the connector of FIG. 1B in a first rotational position.

FIG. 13 illustrates a cross-sectional view of the connector in the first rotational position and taken along the line 12—12 of FIG. 6.

FIG. 14 illustrates a side view of the connector of FIG. 1B in a second rotational position.

FIG. 15 illustrates a cross-sectional view of the connector in the second rotational position and taken along the line 15—15 of FIG. 6.

FIG. 16 illustrates a side view of the connector of FIG. 1B in a third rotational position.

FIG. 17 illustrates a cross-sectional view of the connector in the third rotational position and taken along the line 17—17 of FIG. 6.

FIG. 18 is a flowchart illustrating a method of assembling the rod-to-rod connector, according to some aspects of the present disclosure.

FIG. 19 is a flowchart illustrating a method of use of the rod-to-rod connector, according to some aspects of the present disclosure.

FIG. 20A illustrates a perspective view of a connector according to one embodiment and including a first connector body, a first spinal rod, a second connector body, and bone fastener, according to some aspects of the present disclosure.

FIG. 20B illustrates another perspective view of the connector of FIG. 20A with the first spinal rod removed.

FIG. 21A illustrates an exploded view of the connector of FIG. 20B.

FIG. 21B illustrates an exploded view of the connector of FIG. 20B.

FIG. 22 illustrates a cross-sectional view of the connector of FIG. 20B in an axial position and taken along the line 22—22 of FIG. 20B.

FIG. 23 is a flowchart illustrating a method of use of the connector of FIG. 20A, according to some aspects of the present disclosure.

FIG. 24 illustrates a perspective view of a rod-to-rod connector according to another embodiment and including a first connector body, a first spinal rod, a second connector body, and a second spinal rod, according to some aspects of the present disclosure.

### DETAILED DESCRIPTION

The following description of the depicted embodiment(s) is merely exemplary in nature and is in no way intended to limit the invention, its application, or uses. Before any embodiments of the disclosure are explained in detail, it is to be understood that the present disclosure is not limited in its application to the details of construction and the arrangement of components set forth in the following description or illustrated in the drawings. The present disclosure is capable of other embodiments and of being practiced or of being carried out in various ways.

Embodiments of the presently disclosed system are described in detail with reference to the drawings, in which like reference numerals designate identical or corresponding elements in each of the several views. In the following description, well-known functions or constructions are not described in detail to avoid obscuring the present disclosure in unnecessary detail.

Also, it is to be understood that the phraseology and terminology used herein is for the purpose of description and should not be regarded as limiting. Use of “including” and “comprising” and variations thereof as used herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

The components of the connectors described herein can be fabricated from biologically acceptable materials suitable for medical applications, including metals, synthetic polymers, ceramics and bone material. Examples of such materials include, but are not limited to, a titanium alloy (e.g., Ti-6Al-4V), pure titanium, and cobalt-chromium (CoCr).

In complex spinal procedures, a first spinal rod must be connected to a second spinal rod via a domino connector (e.g., rod-to-rod connector). It is desirable that the rod-to-rod connector be movable in multiple different degrees of freedom during a spinal procedure to allow the surgeon flexibility in aligning multiple screw trajectories across different regions of the spine. For example, in the context of cervical thoracic (CT) procedures, the first spinal rod is coupled to the cervical portion of the spine, while the second spinal rod is coupled to the thoracic portion of the spine. Therefore, the first spinal rod typically may be oriented differently (e.g., have a different trajectory) than the second spinal rod. A rod-to-rod connector that connects the first and second rods desirably considers both sagittal and coronal plane variables. The present disclosure describes a rod-to-rod connector that is translatable to accommodate a variable rod-to-rod distance and is further rotatable to allow for sagittal angle rotation. Additionally, the present disclosure describes a rod-to-rod connector that uses a frictional assembly to assist keeping the rod-to rod-connector in the desired state.

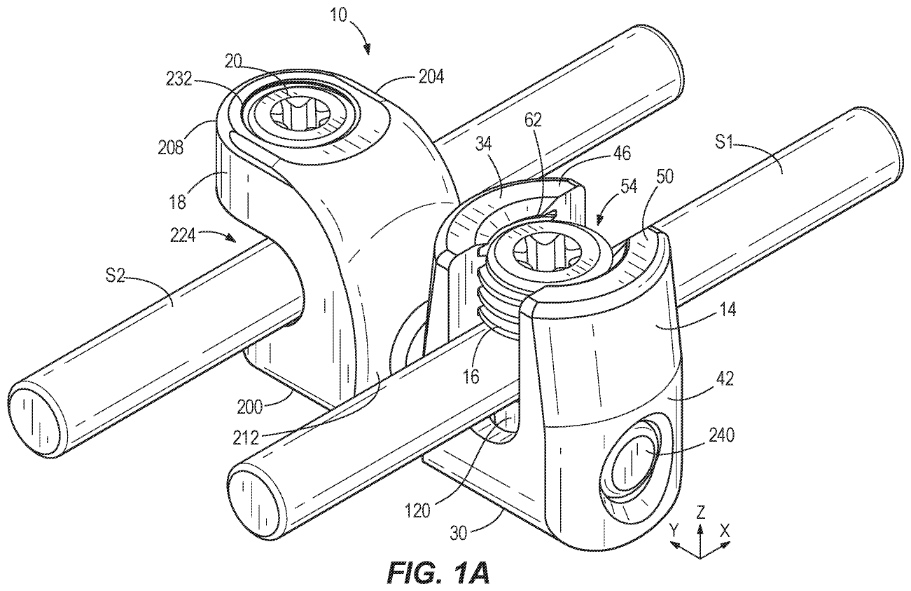

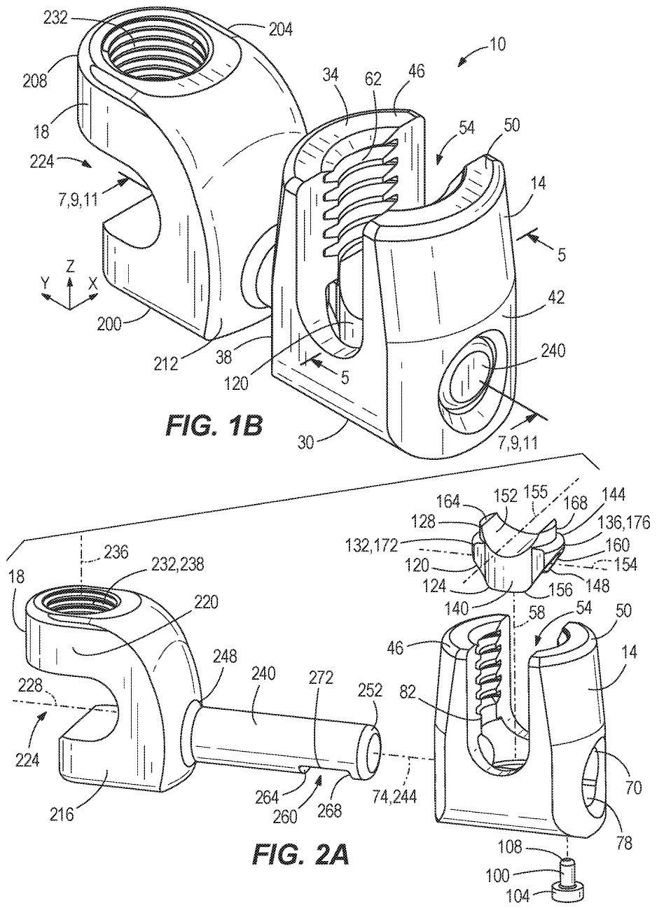

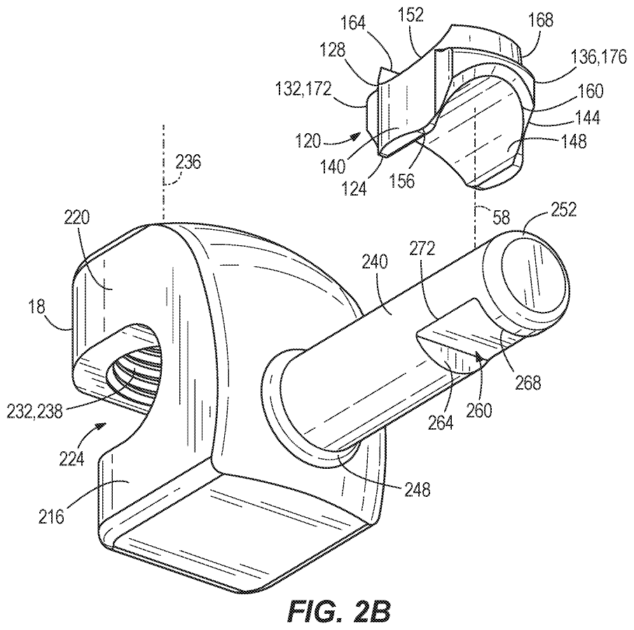

Turning now to FIGS. 1A-2B, a rod-to-rod connector 10 is illustrated according to some aspects of the present disclosure. As shown in FIG. 1A, the connector 10 is configured to couple a first spinal rod S1 to a second spinal rod S2. The connector 10 includes a first connector body 14 and a second connector body 18 that is movably coupled to the first connector body 14. Specifically, a post 240 of the second connector body 18 is received within the first connector body 14. A fastener 100 couples the first connector body 14 to the second connector body 18, while allowing limited movement therebetween. The first connector body 14 is configured to receive and secure the first spinal rod S1 and the second connector body 18 is configured to receive and secure the second spinal rod S2. The first connector body 14 includes a crown 120 that supports the first spinal rod S1. As shown, a threaded fastener 16 couples the first spinal rod S1 to the first connector body 14. The first spinal rod S1 is positioned between the threaded fastener 16 and the crown 120. Similarly, a threaded fastener 20 couples the second spinal rod S2 to the second connector body 18. The first connector body 14 and the second connector body 18 are both translatable and rotatable relative to one another, which allows the connector 10 to accommodate a variable rod-to-rod distance as well as sagittal angle rotation.

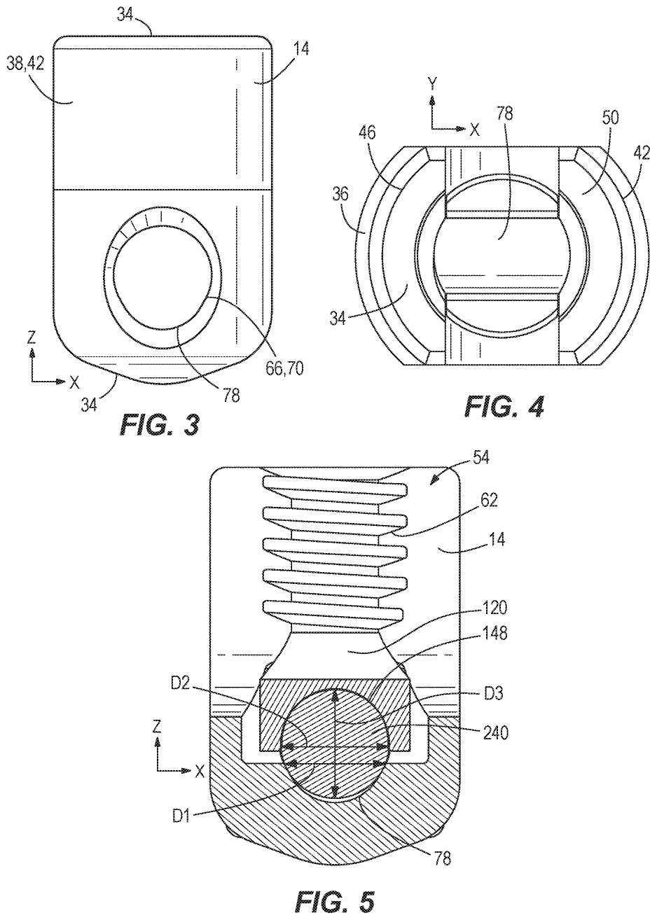

FIGS. 1A-5 illustrate further aspects of the first connector body 14 in greater detail. As shown, the first connector body 14 includes a first end 30, a second end 34, a first side 38, and a second side 42 opposite the first side 38. The first connector body 14 can be formed from Ti-6al-4V, though other biologically acceptable materials are also contemplated. The first connector body 14 is generally U-shaped in the illustrated embodiment to include a first arm 46 and a second arm 50 that is coupled to and spaced apart from the first arm 46. Each of the first arm 46 and the second arm 50 extends from the second end 34 towards the first end 30. A first gap or opening 54 is defined between the first arm 46 and the second arm 50 and extends along a first axis 58. With respect to side and end views in FIGS. 3 and 4, the first arm 46 looks the same as the second arm 50 when viewed from the second end 34, the first side 38, and the second side 42. Together, the first arm 46 and the second arm 50 define a threaded interface 62 as depicted in the perspective view of FIG. 1B and the cross-sectional view of FIG. 5. An inner surface of the first arm 46 defines a first portion of the threaded interface 62 and an inner surface of the second arm 50 defines a second portion of the threaded interface 62. Additional details of the threaded interface 62 are depicted in the cross-sectional view of FIG. 7.

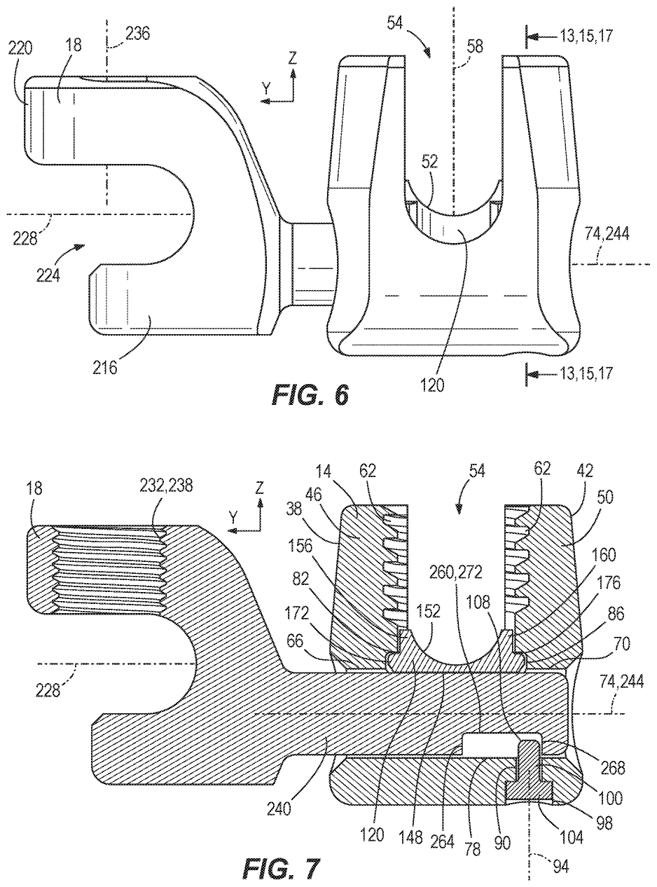

Further with respect to FIG. 3, the first arm 46 further defines a first aperture 66 extending from the first side 38 to the first opening 54 and the second arm 50 defines a second aperture 70 extending from the second side 42 to the first opening 54. As shown in FIGS. 6 and 7, the first aperture 66 and the second aperture 70 are aligned along a second axis 74, which is generally transverse to the first axis 58. The first connector body 14 also has a support recess 78 that extends from the first side 38 to the second side 42 along the second axis 74. The support recess 78 is partially defined by each of the first aperture 66 and the second aperture 70. The support recess 78 defines a first distance D1 (see FIG. 5). As shown in FIG. 5, the first distance D1 is measured between opposite sides of the support recess 78 and transverse to the second axis 74, which is into the page in FIG. 5.

Further with respect to the cross-sectional view of FIG. 7, the first arm 46 defines a first side recess 82 and the second arm 50 defines a second side recess 86. The first side recess 82 and the second side recess 86 are each positioned between the threaded interface 62 and the respective first aperture 66 and second aperture 70.

The first connector body 14 includes an aperture 90 that extends from the first end 30 towards the second end 34. The aperture 90 extends along a third axis 94 that is generally parallel to the first axis 58 and transverse to the second axis 74. The aperture 90 extends into the support recess 78 of the first connector body 14. In the illustrated embodiment, the aperture 90 is positioned such that it also extends into the second aperture 70. In other embodiments, the aperture 70 may be positioned at other locations along a width of the first connector body 14. A bore 98 extends from the first end 30 towards the second end 34 and surrounds the aperture 90. The aperture 90 is configured to receive the fastener 100 that extends therethrough. The fastener 100 includes a first end 104 and a second end 108 opposite the first end 104. When assembled, the first end 104 is seated in the bore 98, while the second end 108 extends into the support recess 78. In the illustrated embodiment, the fastener 100 is a pin that is welded to the first connector body 14. In other embodiments, the fastener 100 may have other configurations, such as a threaded fastener or a detent mechanism. Additionally, the fastener 100 can be formed from Ti-6A1-4V, though other biologically acceptable materials may also be used.

Returning to FIGS. 1A-2B, the crown 120 is shown in greater detail. The crown 120 is supported by the first connector body 14 and is configured to support the first spinal rod S1 within the first opening 54. The crown 120 can be formed from pure titanium, though other biologically acceptable materials may also be used. The crown 120 includes a first end 124, a second end 128 opposite the first end 124, a first side 132, a second side 136 opposite the first side 132, a third side 140, and a fourth side 144 opposite the third side 140. The crown 120 includes a first crown recess 148 defined in the first end 124 and a second crown recess 152 defined in the second end 128. The first crown recess 148 extends from the first side 132 to the second side 136 along a first longitudinal axis 154. As shown, the first crown recess 148 is positioned between the third side 140 and the fourth side 144. The second crown recess 152 is positioned between the first side 132 and the second side 136 along a second longitudinal axis 155. The second crown recess 152 extends between the third side 136 and the fourth side 144. The first crown recess 148 (and the first longitudinal axis 154 thereof) is oriented generally transverse to the second crown recess 152 (and the second longitudinal axis 155 thereof). First and second arms 156, 160 extend from the first end 124 towards the second end 128. The first and second arms 156, 160 define the first crown recess 148. The first crown recess 148 has a second distance D2 (FIG. 5). As shown in FIG. 5, the second distance D2 is measured between opposite sides of the first crown recess 148 and transverse to the longitudinal axis 154, which is into the page in FIG. 5. Third and fourth arms 164, 168 extend from the second end 128 toward the first end 124. The third and fourth arms 156, 160 define the second crown recess 152. A first crown projection 172 is positioned on the first side 132. A second crown projection 176 is positioned on the second side 136.

The crown 120 is positioned within the first connector body 14 such that the first crown recess 148 faces the support recess 78 and the second crown recess 152 faces the second end 34 of the first connector body 14. When assembled, the first crown projection 172 is received within the first side recess 82 of the first arm 46 (as also depicted in FIG. 7), such that the third arm 164 of the crown 120 is positioned adjacent to the first arm 46 of the first connector body 14. Similarly, the second crown projection 176 is received within the second side recess 86 of the second arm 50 (as also depicted in FIG. 7), such that the fourth arm 168 of the crown 120 is positioned adjacent to the second arm 50 of the first connector body 14.

The second connector body 18 includes a first end 200, a second end 204 opposite the first end 200, a first side 208, and a second side 212 opposite the first side 208. In the illustrated embodiment, the second connector body 18 is formed from Ti-6A1-4V, though other biologically acceptable materials may also be used. The second connector body 18 includes a first arm 216 and a second arm 220 that is coupled to and spaced apart from the first arm 216. As shown, the first arm 216 and the second arm 220 each extend from the first side 208 towards the second side 212. A second gap or opening 224 is defined between the first arm 216 and the second arm 220 and extends along a third axis 228 (also shown in FIG. 6). The second opening 224 is configured to receive the second spinal rod S2. The second arm 220 includes an aperture 232 extending therethrough from the second end 204 toward the second opening 224. The aperture 232 extends along a fourth axis 236 and has a threaded interface 238.

A post 240 extends laterally from the second side 212 of the second connector body 18 along a fifth axis 244. In the illustrated embodiment, the fifth axis 244 is generally parallel to the third axis 228. In the illustrated embodiment, the post 240 is integrally formed with the second connector body 18, but in other embodiments the post 240 may be coupled to the second connector body 18 in other ways, such as welding or fasteners. The post 240 is generally cylindrical and has a third diameter D3 (FIG. 5). The post 240 has a first end 248 and a second end 252 opposite the first end 248. The first end 248 is integrally formed with (or otherwise coupled to) the second side 212 of the second connector body 18. The post 240 also has a groove 260. In the illustrated embodiment, the groove 260 is positioned closer to the second end 252 of the post 240, and more specifically, adjacent to the second end 252 of the post 240. The groove 260 includes a first end 264, a second end 268 opposite the first end 264, and a recessed surface 272 extending between the first end 264 and the second end 268. The first end 264 of the groove 260 is positioned closer to the first end 248 of the post 240 than the second end 252, while the second end 268 of the groove 260 is positioned closer to the second end 252 of the post 240 than the first end 248.

Turning now to FIGS. 5-11, the first connector body 14 and the second connector body 18 are movably coupled together via the post 240 and the fastener 100 (see FIGS. 7, 9, and 11). The post 240 is received within the first connector body 14. The post 240 extends at least partially through the first connector body 14 such that the second connector body 18 is positioned on the first side 30 of the first connector body 14 and the groove 260 in the post 240 overlaps the aperture 90 in the first connector body 14. Also, the post 240 is positioned between the first connector body 14 and the crown 120. More specifically, the post 240 is at least partially positioned within the first aperture 66 and the second aperture 70, such that the post 240 is positioned within the support recess 78 of the first connector body 14 and the first crown recess 148 of the crown 120. With specific reference to FIG. 5, the first distance D1 of the support recess 78 in the first connector body 14 and the second distance D2 of the first crown recess 148 of the crown 120 are both smaller than the third diameter D3 of the post 240. This allows an interference fit to be created between the crown 120, and the post 240 in an unlocked state. This allows a taper locked condition to be created between the first connector body 14 and the post 240 in a permanently coupled state, as will be discussed below. With specific reference to FIGS. 7, 9, and 11, the groove 260 of the post 240 overlaps the aperture 90 of the first connector body 14. Therefore, the second end 108 of the fastener 100 is positioned within the groove 260.

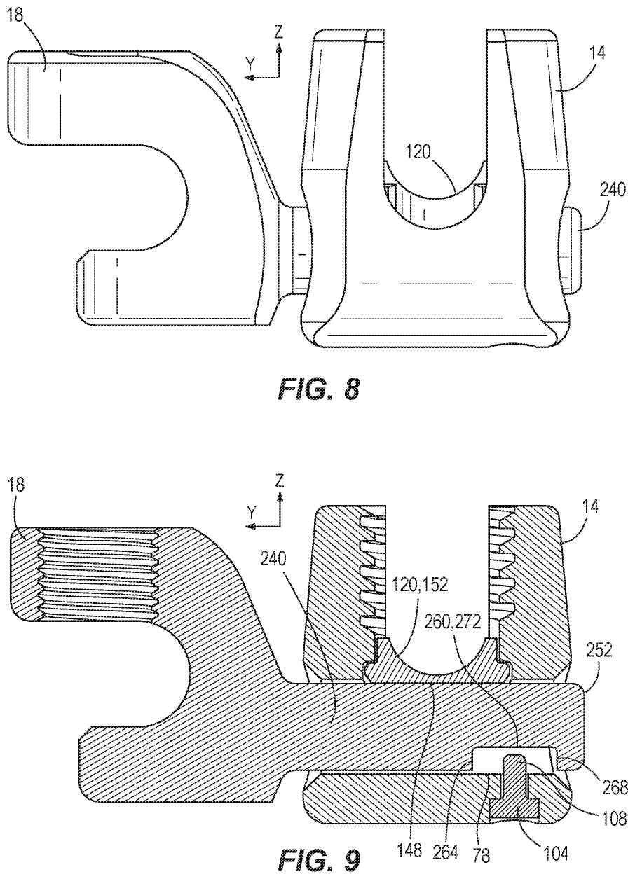

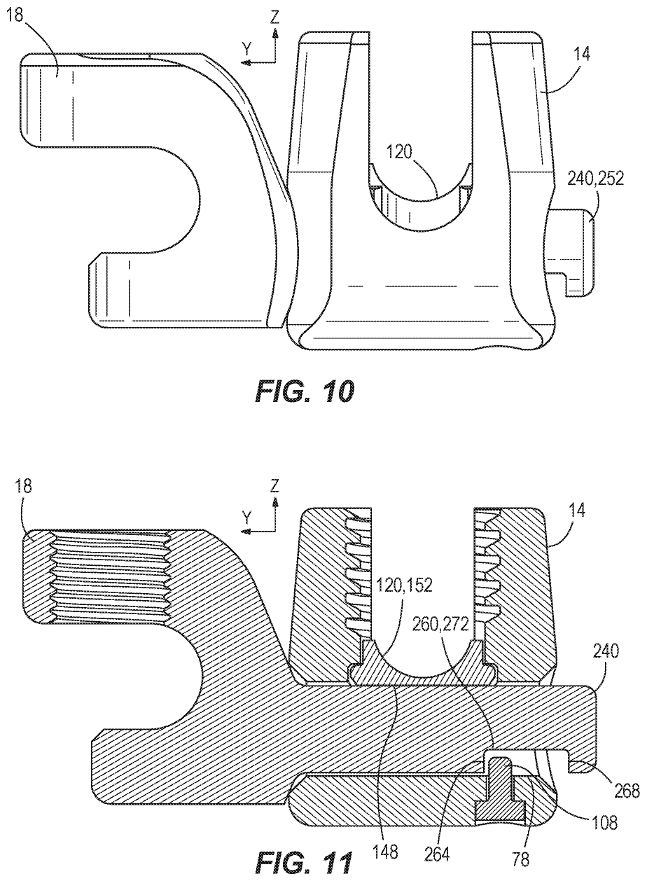

Further with respect to FIGS. 6-11, the first connector body 14 and the second connector body 18 are axially or translationally movable relative to one another to change a distance between the first opening 54 and the second opening 224. By allowing this desirable movement, the first and second openings 54, 224 can more easily accommodate the distances between the first and second spinal rods S1, S2. As shown, the second connector body 18 is movable between a first axial position (FIGS. 6 and 7) and a second axial position (FIGS. 10 and 11). Additionally, the second connector body 18 can be moved to a plurality of intermediate axial positions between the first and second axial positions. One of the intermediate axial positions is shown in FIGS. 8 and 9. Because of the interference fit between the crown 120 and the post 240, in the unlocked state, the second connector body 18 can temporarily maintain its axial position relative to the first connector body 14. For example, due to the interference fit, the first connector body 14 and the second connector body 18 will not move relative to one another in any of the axial positions prior to being permanently coupled relative to one another (as will be discussed in greater detail below).

Referring to FIGS. 6 and 7, in the first axial position, the second connector body 18 is spaced apart from the first connector body 14. Accordingly, a gap exists between the first connector body 14 and the second connector body 18. Also, in the first axial position, the second end 108 of the fastener 100 is seated against the second end 268 of the groove 260.

Referring to FIGS. 8 and 9, in the intermediate axial positions, the second connector body 18 is spaced apart from the first connector body 14 but by a smaller distance than in the first axial position. Accordingly, a gap exists between the first connector body 14 and the second connector body 18 that is smaller than the gap of the first axial position. Also, in the intermediate axial positions, the second end 108 of the fastener 100 is positioned between the first end 264 and the second end 268 of the groove 260.

Referring to FIGS. 10 and 11, in the second axial position, the second connector body 18 abuts the first connector body 14. Also, in the second axial position, the second end 108 of the fastener 100 is seated against the first end 264 of the groove 260. The second end 252 of the post 240 extends from the second side 42 of the first connector body 14, as well.

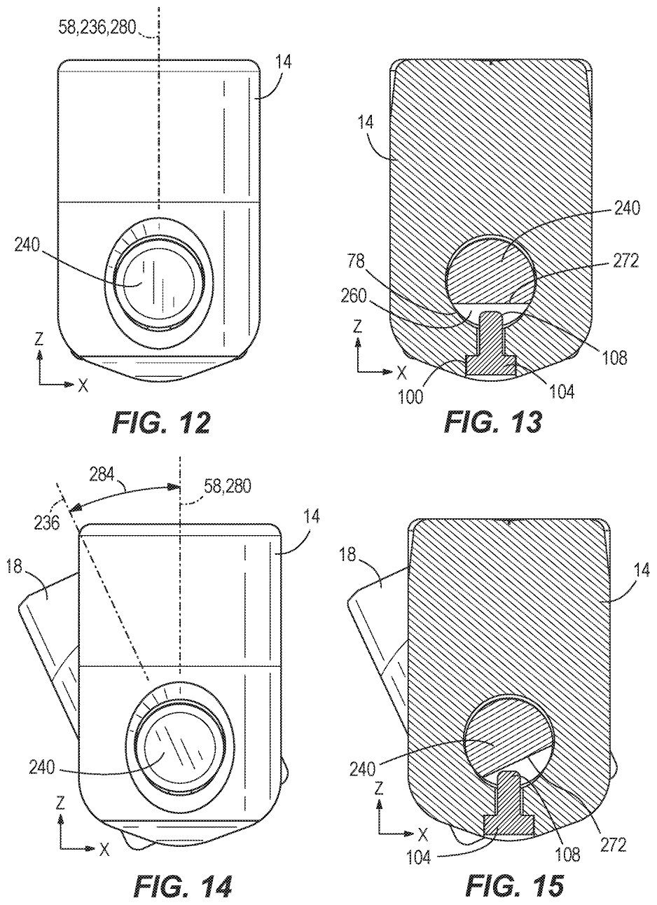

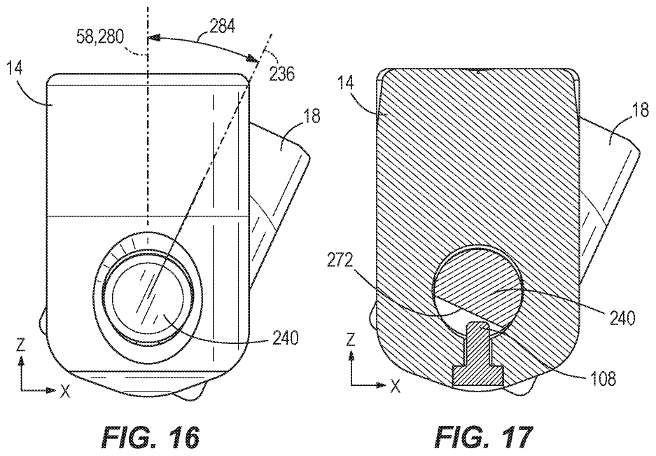

Referring to FIGS. 12 to 17, the first connector body 14 and the second connector body 18 are also rotationally movable or pivotable relative to one another to adjust the relative angular position of the first opening 54 and the second opening 224 relative to one another. Accordingly, the first and second openings 54, 224 can more easily fit the relative angular positions of the first and second spinal rods S1, S2. As shown, the second connector body 18 is movable from a first rotational position (FIGS. 12 and 13) to either a second rotational position (FIGS. 14 and 15) or to a third rotational position (FIGS. 16 and 17). Additionally, the second connector body 18 can be moved to a plurality of intermediate rotational positions between the first rotational position and the second rotational position and between the first rotational position and the third rotational position. Again, because of the interference fit between the crown 120 and the post 240, in the unlocked state, the second connector body 18 can temporarily maintain its rotational position relative to the first connector body 14. For example, due to the interference fit, the first connector body 14 and the second connector body 18 will not move relative to one another in any of the rotational positions prior to being permanently coupled relative to one another (as will be discussed in greater detail below).

In the first rotational position, the second connector body 18 is generally aligned with the first connector body 14. For example, the fourth axis 236 of the second connector body 18 is generally parallel to the first axis 58 of the first connector body 14. Moreover, the fourth axis 236 extends within a plane 280 defined by the first axis 58 and the second axis 74 (see FIG. 2A for second axis 74).

In the second rotational position, the second connector body 18 has now been rotated in a first direction relative to the first connector body 14. Accordingly, the second connector body 18 rotates or is offset from the first connector body 14 in the first direction. In the third rotational position, the second connector body 18 has now been rotated in a second direction relative to the first connector body 14. Accordingly, the second connector body 18 rotates or is offset from the first connector body 14 in the second direction. In each of the second and third rotational positions, the fourth axis 236 of the second connector body 18 is rotated relative to the first axis 58 of the first connector body 14. Moreover, the fourth axis 236 is positioned at a non-parallel angle 284 relative to the plane 280 defined by the first axis 58 and the second axis 74 from the first rotational position.

In the illustrated embodiments of FIGS. 12 to 17, the non-parallel angle 284 measures substantially 30 degrees in each of the second and third rotational positions. Accordingly, the second connector body 18 is movable relative to the first connector body 14 within a substantially 60-degree range (e.g., along a substantially 60-degree arc). In other embodiments, the non-parallel angle may range from substantially 10 degrees to substantially 90 degrees. Accordingly, the second connector body 18 may be movable relative to the first connector body 14 from within a substantially 20-degree range (e.g., along a substantially 20-degree arc) to within a substantially 180-degree range (e.g., along a substantially 180-degree arc). In other embodiments, the non-parallel angle in each of the first and second rotational positions may be substantially 10 degrees, substantially 20 degrees, substantially 30 degrees, substantially 40 degrees, substantially 50 degrees, substantially 60 degrees, substantially 70 degrees, substantially 80 degrees, or substantially 90 degrees. Additionally, although the non-parallel angle in the illustrated embodiment is the same on both sides of the connector 10, in other embodiments, the non-parallel angle on the first side of the connector 10 may be different than the non-parallel angle on the second side of the connector 10. Regardless, the second connector body 18 may be movable relative to the first connector body 14 within the range defined by the non-parallel angles defined in the second and third rotational positions. The second connector body 18 is prevented from rotating beyond the second and third rotational positions via engagement between the recessed surface 272 of the groove 260 and second end 108 of the fastener 100. Therefore, the fastener 100 and the post 240 collectively define a stop.

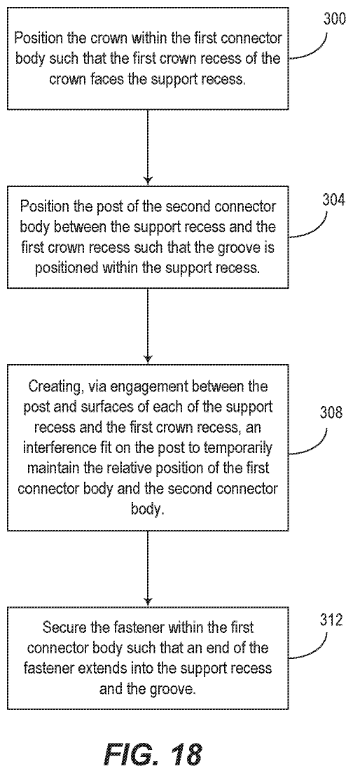

In some aspects, the connector 10 can be assembled using the following operational steps, as shown in FIG. 18. For example, at operational step 300, the crown 120 is positioned within the first connector body 14 such that the first crown recess 148 of the crown 120 faces the support recess 78. The crown 120 is inserted into first connector body 14. Specifically, while the crown 120 is oriented such that the first crown recess 148 (e.g., the longitudinal axis 154 thereof) extends generally transverse to the second axis 74, the crown 120 is axially inserted into the first opening 54 along the first axis 58. In other words, while the crown 120 is being inserted into the first opening 54, the first side 132 and the second side 136 of the crown 120 are positioned between the first arm 46 and the second arm 50, respectively. Following the crown 120 being positioned within the first opening 54, the crown 120 is then rotated about the first axis 58 by approximately 90 degrees such that the first crown projection 172 is received in the first side recess 82 of the first arm 46 and the second crown projection 176 is received in the second side recess 86 of the second arm 50. Also, rotating the crown 120 by approximately 90 degrees orients the support recess 78 (e.g., the longitudinal axis 154 thereof) generally parallel to the second axis 74. Next, in operational step 304, once the crown 120 is in place, the post 240 is positioned within the first connector body 14 between the support recess 78 thereof and the first crown recess 148 of the crown 120. Positioning the post 240 within the first connector body 14 also overlaps the aperture 90 with the groove 260. Next, in operational step 308, a surface of the support recess 78 of the first connector body 14 and a surface of the first crown recess 148 of the crown 120 create an interference fit against the post 240. Then, in operational step 312, with the groove 260 overlapping the aperture 90, the fastener 100 is inserted into the aperture 90 such that the first end 104 is seated in the bore 98 and the second end 108 extends into the support recess 78 and the groove 260. The fastener 100 is permanently coupled (e.g., secured) to the first connector body 14. In the illustrated embodiment, the fastener 100 can be welded to the first connector body 14.

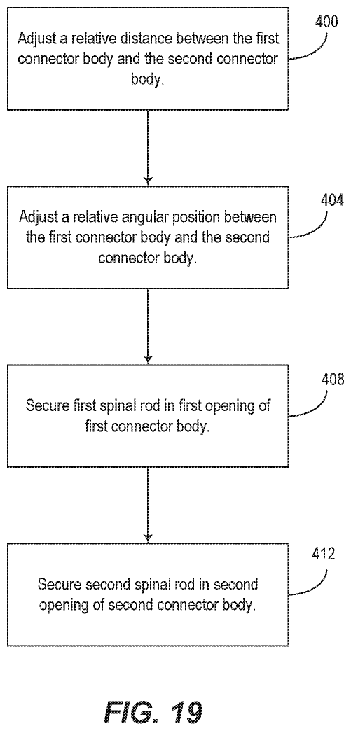

As noted above, the connector 10 is configured to receive and secure the first spinal rod S1 and the second spinal rod S2. Referring to FIG. 19, an exemplary method of use of the present rod-to-rod connector is described. For example, once the first and second spinal rods S1, S2 are appropriately secured to the vertebral members, at operational steps 400 and 404, the user may adjust the relative distance and angular position of the first and second connector bodies 14, 18 to the relative positions of the first spinal rod S1 and the second spinal rod S2. As noted above, the interference fit between the second connector body 18 and the crown 120 enables the first and second connector bodies 14, 18 to temporarily maintain the relative distance and angular position. In some embodiments, either the distance or the angular position may be adjusted rather than both. At operational step 408, the user may secure the first spinal rod S1 within the first opening 54 of the first connector body 14 to achieve the permanently coupled state. In particular, the first spinal rod S1 may be positioned within the first opening 54 of the first connector body 14 and supported by the second crown recess 152 of the crown 120. Then, the user may couple the threaded fastener 16 within the first opening 54 to secure the first spinal rod S1 therein. In coupling the threaded fastener 16 within the first opening 54, the threaded fastener 16 exerts a force on the first spinal rod S1, which exerts a force on the crown 120. The force on the crown 120 causes a force on the post 240, which causes further engagement of the post 240 with the surfaces of both the first crown recess 148 of the crown 120 and the support recess 78 of the first connector body 14 thereby creating a taper lock between the first connector body 14, the crown 120, and the post 240. The threaded fastener 16 retains the first spinal rod S1 within the first opening 54, while the taper lock prevents relative movement between the first connector body 14 and the second connector body 18. Similarly, at operational step 412, the second spinal rod S2 may be positioned within the second opening 224 of the second connector body 18. The user may then couple the threaded fastener 20 within the second opening 224 to secure the second spinal rod S2 therein. The threaded fastener 20 may exert a force on the second spinal rod S2 to retain the second spinal rod S2 within the second opening 224. In other embodiments, the second spinal rod S2 may be positioned and secured within the second opening 224 before the first spinal rod S1 is positioned and secured within the first opening 54. In other words, operational steps 408 and 412 may be inverted.

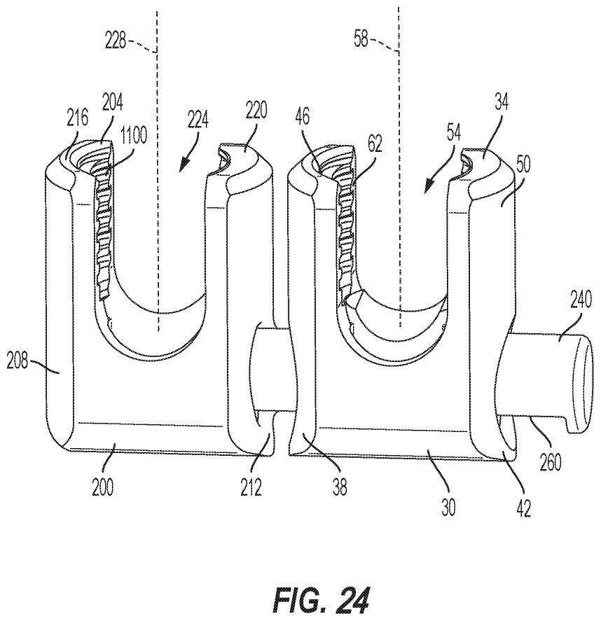

In some aspects of the present disclosure, the illustrated first connector body 14 can be considered a “closed-ended” connector because the first opening 54 is closed via the threaded fastener. The illustrated second connector body 18 can be considered an “open-ended” connector because even after the threaded fastener is installed, the second opening 224 remains open. In some implementations, the first connector body 14 may be an “open-ended” connector and the second connector body 18 may be a “closed-ended” connector. In other implementations, the first connector body 14 and the second connector body 18 may both be open-ended or both be closed-ended (e.g., as shown in FIG. 24).

In exemplary FIG. 24, the second connector body 18, like the first connector body 14, is generally U-shaped in the illustrated embodiment to include a first arm 216 and a second arm 220 that is coupled to and spaced apart from the first arm 216. Each of the first arm 216 and the second arm 220 extends from the second end 204 towards the first end 200. A first gap or opening 224 is defined between the first arm 216 and the second arm 220 and extends along a third axis 228. Together, the first arm 216 and the second arm 220 define a threaded interface 1100. An inner surface of the first arm 216 defines a first portion of the threaded interface 1100 and an inner surface of the second arm 220 defines a second portion of the threaded interface 1100. As discussed above, the post 240 extends laterally from the second side 212 of the second connector body 18 along the fifth axis 244. The details of the post 240 are the same as the second connector body 18 of FIGS. 1-17.

As shown, whereas the third axis 228 of the second connector body 18 of FIGS. 1-17 is generally parallel to the fifth axis 244 of the post 240, the third axis 228 of the second connector body 18 of FIG. 24 is transverse (e.g., perpendicular) to the fifth axis 244 of the post 240. Accordingly, the third axis 228 of FIG. 24 is oriented like the fourth axis 236 of FIGS. 1-17. The relative sliding movement between the first connector body 14 and the second connector body 18 of FIG. 24 is generally the same as the relative movement of the first connector body 14 and the second connector body 18 of FIGS. 1-17. The relative rotational movement between the first connector body 14 and the second connector body 18 of FIG. 24 is generally similar as the relative movement of the first connector body 14 and the second connector body 18 of FIGS. 1-17. For clarity the details are discussed below.

In the first rotational position, the second connector body 18 is generally aligned with the first connector body 14. For example, the third axis 228 of the second connector body 18 is generally parallel to the first axis 58 of the first connector body 14. Moreover, the third axis 228 extends within the plane 280 defined by the first axis 58 and the second axis 74.

In the second rotational position, the second connector body 18 is rotatable in the first direction relative to the first connector body 14. Accordingly, the second connector body 18 rotates or is offset from the first connector body 14 in the first direction. In the third rotational position, the second connector body 18 is rotatable in the second direction relative to the first connector body 14. Accordingly, the second connector body 18 rotates or is offset from the first connector body 14 in the second direction. In each of the second and third rotational positions, the third axis 228 of the second connector body 18 is rotated relative to the first axis 58 of the first connector body 14. Moreover, the third axis 228 may be positioned at a non-parallel angle 284 relative to the plane 280 defined by the first axis 58 and the second axis 74 from the first rotational position.

In another aspect of the present disclosure, the illustrated first connector body 14 and the illustrated second connector body 18 of FIGS. 1-17 and 24 can alternatively be considered “open-ended” connectors. That is, the first connector body 14 and the second connector body 18 are both open-ended connectors because the rods are inserted into the connectors 14, 18 via the respective openings 54, 224. In some embodiments, the first connector body and the second connector body can alternatively be considered “closed-ended” connectors. That is, rather than having openings 54, 224, the first and second connector bodies 14, 18 may have apertures that receive the rods. That is, the openings 54, 224 may be fully enclosed (not shown) by the respective first and second connector bodies 14, 18. Each of the apertures may be in communication with threaded apertures that receive threaded fasteners, which secure the respective rod within the respective aperture. In such case, the rods would slide into the apertures via a third or fourth side of the respective connector body 14, 18. In some embodiments, the first connector body 14 may be closed-ended and the second connector body 18 may be open-ended. In still other embodiments, the first connector body 14 may be open-ended and the second connector body 18 may be closed-ended.

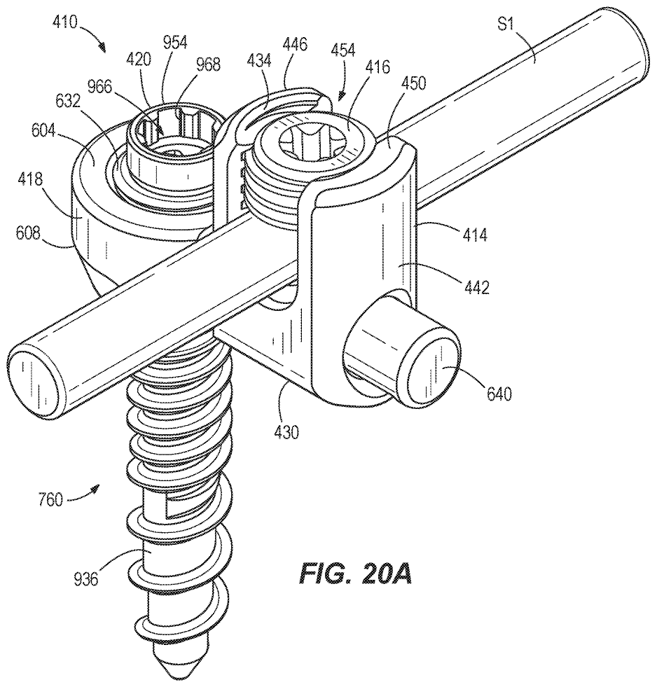

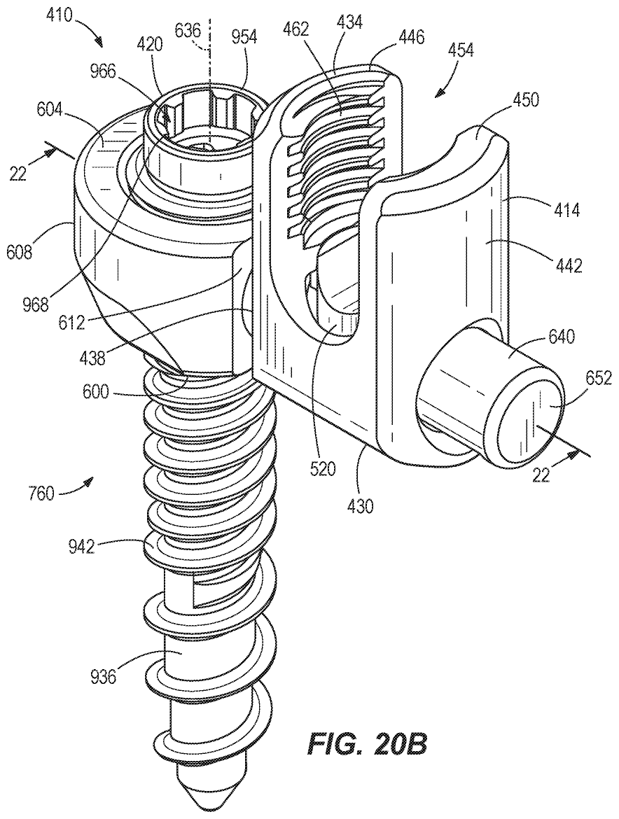

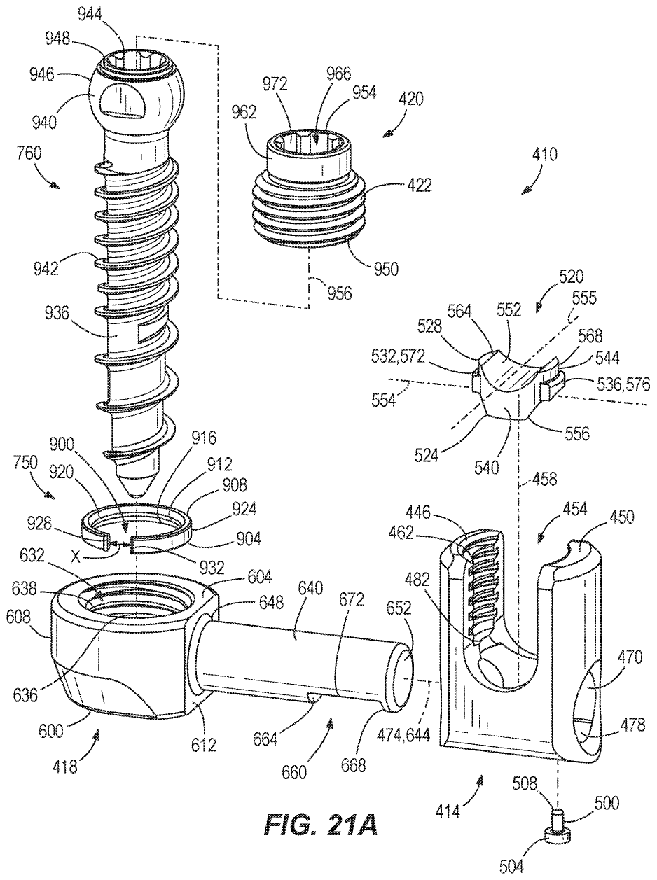

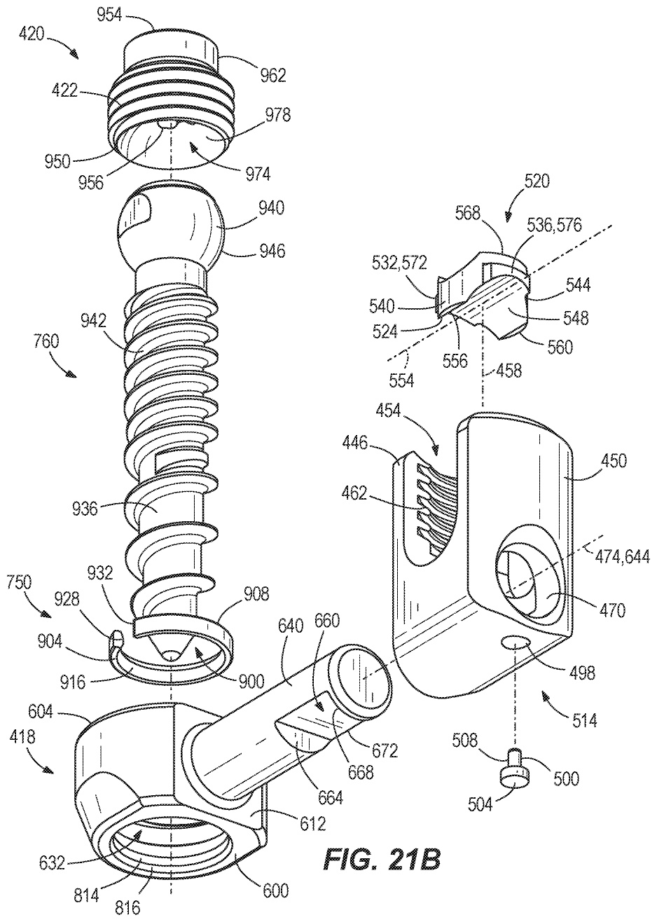

Turning now to FIGS. 20A-22, a bone fastener-to-rod connector 410 is illustrated according to some aspects of the present disclosure. The connector 410 is similar to the connector 10 of FIGS. 1-17. Therefore, like structure will be identified using the same reference numerals plus “400” and only the differences will be discussed herein. The first connector body 414 of FIGS. 20A-22 includes the same features as the first connector body 14 of FIGS. 1-17. In the embodiment of FIGS. 20A-22, a second connector body 418 may act as a receiver. A retainer 750 (FIG. 21A) is positioned within the second connector body 418, and a bone screw 760 is coupleable to the second connector body 418 using a threaded fastener 420 and the retainer 750.

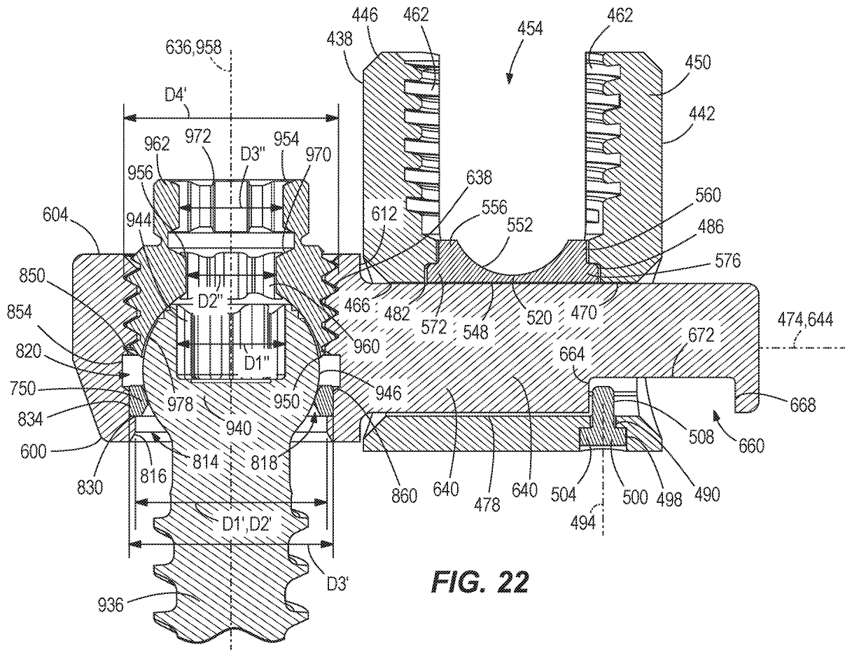

The second connector body 418 includes a first end 600, a second end 604 opposite the first end 600, a first side 608, and a second side 612 opposite the first side 608. In the illustrated embodiment, the second connector body 418 is formed from Ti-6A1-4V, though other biologically acceptable materials may also be used. An aperture 632 of the second connector body 418 extends between the first end 600 and the second end 604. As shown in FIG. 22, a threaded interface 638 of the aperture 632 extends partially through the aperture 632 from the second end 604 toward the first end 600.

Further with respect to FIG. 22, the aperture 632 defines an opening 814 at the first end 600. As shown, the opening 814 extends from the first end 600 toward the threaded interface 638. The opening 814 defines a first dimension D1′ (e.g., inner diameter) that is substantially the same as a second dimension D2′ (e.g., inner diameter) of the threaded interface 638. In the illustrated embodiment, a beveled surface 816 partially defines the opening 814. The beveled surface 816 is positioned at the first end 600. The second connector body 418 includes a first circumferential recess or groove 818 and a second circumferential recess or groove 820, each of which is concentric with the aperture 632. As shown, the grooves 818, 820 open towards the aperture 632.

Still referring to FIG. 22, the first groove 818 is defined by a continuous annular surface 830 located above and adjacent to the opening 814 and a cylindrical surface 834 that is located above and adjacent to the surface 830. The surface 830 is generally perpendicular to the surface 834. As shown, the surface 830 defines a ledge. The cylindrical surface 834 is oriented substantially parallel to a fourth axis 636 and defines a third dimension (e.g., inner diameter) D3′, which is greater than the first dimension D1′. The second groove 820 is defined by a continuous annular surface 850 located below and adjacent to the threaded interface 638 and a cylindrical surface 854 that is located below and adjacent to the surface 850. The surface 850 is generally perpendicular to the surface 854. As shown, the surface 850 defines a ledge. The cylindrical surface 854 is oriented substantially parallel to the fourth axis 636 and defines a fourth dimension (e.g., diameter) D4′, which greater than the first dimension D1′ of the opening 814 and the third dimension D3′ of the first groove 816. The surface 854 is coupled to the surface 834 by a beveled surface 860.

Like the second connector body 418 of FIGS. 1-17, a post 640 extends laterally from the second side 612 of the second connector body 418 of FIGS. 20A-22 and includes the features of the post 240 described above. Accordingly, the first connector body 414 and the second connector body 418 are movably coupled together via the post 640 and a fastener 500 (in the same way as discussed above for FIGS. 1-17).

In the illustrated embodiment, the retainer 750 is a resilient retainer. The retainer 750 is made from a resilient material, such as a stainless steel or titanium alloy, so that the retainer 750 may be both compressed and expanded during various steps of assembly as will be described in greater detail below. As shown in FIGS. 21A and 21B, the retainer 750 has a central channel or hollow through bore, generally 900, that passes entirely through the structure from a first surface 904 to a second, opposite surface 908 thereof. Surfaces that define the channel or bore 900 include a discontinuous inner cylindrical surface 912 positioned between a first discontinuous frusto-conical or beveled surface 916 and a second discontinuous frusto-conical or beveled surface 920. Each of the surfaces 912, 916, 920 is coaxial with the fourth axis 636 when the retainer 750 is in a neutral non-compressed, non-expanded orientation. The first beveled surface 916 is adjacent the first surface 904 and the second beveled surface 920 is adjacent the second surface 908. The retainer 750 further includes an outer cylindrical surface 924 located adjacent the first and second surfaces 904, 908. The surface 924 is oriented parallel to the fourth axis 436. The resilient retainer 750 further includes first and second end surfaces 928, 932 disposed in spaced relation to one another when the retainer 750 is in a neutral non-compressed state. Both end surfaces 928, 932 are disposed substantially perpendicular to the first and second surfaces 904, 908. A width X (FIG. 21A) between the surfaces 928, 932 is determined by a desired amount of compressibility of the open retainer 750 when loaded into the second connector body 418.

The width X provides adequate space between the surfaces 928, 932 for the retainer 750 to be pinched, with the surfaces 928, 932 compressed toward one another to a closely spaced or even touching configuration, if necessary, to an extent that the compressed retainer 750 is up or bottom loadable through the second connector body opening 814. After passing through the opening 814, the retainer 750 expands or springs back to an original uncompressed, rounded or collar-like configuration and can be seated within the first groove 818. The embodiment herein illustrates the surfaces 928, 932 as substantially parallel, however, it is foreseen that it may be desirable to orient the surfaces obliquely and/or at a slight angle depending upon the amount of compression desired during loading of the retainer 750 into the second connector body 418.

With particular reference to FIG. 21A, the bone screw 760 is configured to be received within and coupled to the second connector body 418. The bone screw 760 includes a shaft 936 and a head 940. The shaft 936 is configured to penetrate tissue, such as, for example, a vertebral member. In some embodiments, the shaft 936 includes an outer surface having an external threaded interface 942. In some embodiments, the external threaded interface 942 may include a single thread turn or a plurality of discrete threads. The head 940 includes a tool engaging portion 944 (e.g., an inner drive) configured to engage a surgical tool or instrument, as described herein. The inner drive 944 defines a first inner drive dimension D1″ (FIG. 22). In some embodiments, the inner drive 944 includes a hexagonal cross-section. In some embodiments, the inner drive 944 may have alternative cross-sections, such as, for example, rectangular, polygonal, hexalobe, oval, or irregular. The head 940 includes an outer surface 946 that defines a plurality of ridges 948 to improve engagement of the head 940 with the threaded fastener 420 as will be discussed in greater detail below. In some embodiments, the outer surface 946 may include planar surfaces or flats and/or arcuate surfaces instead of the plurality of ridges 948.

The threaded interface 638 is configured to matingly engage with a threaded interface 422 of the threaded fastener 420. As shown in FIGS. 21A-22, the threaded fastener 420 includes a first end 950 and a second, opposite end 954. The threaded interface 422 extends from the first end 950 towards the second end 954. The threaded fastener 420 includes an aperture 956 (FIG. 22) extending therethrough. A sixth axis 958 extends between the first end 950 and the second end 954 and through the aperture 956. The sixth axis 958 is configured to be aligned with the fourth axis 436 when the threaded fastener 420 is coupled to the second connector body 418. The aperture 956 defines a tool-engaging portion 960 (e.g., an inner drive) having a second inner drive dimension D2″ (FIG. 22). The second inner drive dimension D2″ may be the same as or different than the first inner drive dimension D1″ of the inner drive 944 of the bone screw 760. In the illustrated embodiment, like the inner drive 944 of the bone screw 760, the inner drive 960 of the threaded fastener 420 has a hexagonal cross-section, but may have other cross-sectional shapes, as noted above. The second inner drive D2″ is configured to engage another surgical tool or instrument to facilitate revision of the threaded fastener 420.

A wall 962 extends from the second end 954 toward the first end 950 and circumscribes the aperture 956. The wall 962 defines an opening 966 (FIG. 21A) that extends from the second end 954 toward the first end 950. As shown in FIG. 22, a lip 970 is defined between the aperture 956 and the wall 962. The wall 962 also defines a tool-engaging portion 972 (e.g., an inner drive), which has a third inner drive dimension D3″ that is greater than the second inner drive dimension D2″ of the aperture 956. The inner drive 972 of the wall 962 is configured to engage another surgical tool or instrument to facilitate breakoff of the threaded fastener 420.

A cavity 974 extends from the first end 950 toward the second end 954 and has a generally dome-shaped or spherically-shaped inner surface 978 (FIGS. 21B and 22). The aperture 956 is in communication with the cavity 974. The cavity 974 is configured to matingly receive the head 940 of the bone screw 760 such that the inner drive 944 of the bone screw 760 aligns with the inner drive 960 of the threaded fastener 420. When assembled, the bone screw 760 is locked within the second connector body 418 via engagement of the head 940 with the threaded fastener 420 and the retainer 750. That is, the head 940 of the bone screw 760 is positioned and may be locked between the inner surface 978 of the cavity 974 of the threaded fastener 420 and the retainer 750. As shown in FIG. 22, the head 940 may contact the inner surface 978 while the threaded fastener 420 is coupled to the second connector body 418 and may also contact the retainer 750, which is seated in the first groove 818.

The first connector body 414 and the second connector body 418 of FIGS. 20A-22, like the first connector body 14 and the second connector body 18 of FIGS. 1-17, are axially or translationally movable relative to one another to change a distance between a first opening 454 and the bone screw 760. By allowing this desirable movement, the first opening 454 and the bone screw 760 can more easily accommodate the distances between the first spinal rod S1 and the bone screw 760. Accordingly, the surgical staff is able to adjust the connector 410 to accommodate for the location of the spinal rod S1, rather than adjusting the location of the spinal rod S1 to match the location and trajectory of the bone screw 760. Moreover, as discussed with respect to FIGS. 1-17, the first connector body 414 is movable between a first axial position (similar to FIGS. 6 and 7) and a second axial position (FIG. 22). Additionally, the first connector body 414 can be moved to a plurality of intermediate axial positions between the first and second axial positions. One of the intermediate axial positions is similar to that shown in FIGS. 8 and 9. Because of an interference fit between a crown 520 and the post 640, in the unlocked state, first and second connector bodies 414, 418 can temporarily maintain their axial positions relative to one another. For example, due to the interference fit, the first connector body 414 and the second connector body 418 will not move relative to one another in any of the axial positions prior to being permanently coupled relative to one another. The connector 410 of FIGS. 20A-22 is therefore able achieve the same axial positions as the connector 10, as discussed above and shown in FIGS. 6-11.

The first connector body 414 and the second connector body 418 of FIGS. 20A-22, like the first connector body 14 and the second connector body 18 of FIGS. 1-17, are also rotationally movable or pivotable relative to one another to adjust the relative angular position of the first opening 454 and bone screw 760 relative to one another. Accordingly, the first opening 454 can more easily fit the relative angular positions of the first spinal rod S1. The first connector body 414 is movable from a first rotational position (similar to that of FIGS. 12 and 13) to either a second rotational position (similar to that of FIGS. 14 and 15) or to a third rotational position (similar to that of FIGS. 16 and 17). Additionally, the first connector body 414 can be moved to a plurality of intermediate rotational positions between the first rotational position and the second rotational position and between the first rotational position and the third rotational position. Again, because of the interference fit between the crown 520 and the post 640, in the unlocked state, first and second connector bodies 414, 418 can temporarily maintain their rotational positions relative to one another. For example, due to the interference fit, the first connector body 414 and the second connector body 418 will not move relative to one another in any of the rotational positions prior to being permanently coupled relative to one. The connector 410 of FIGS. 20A-22 is therefore able achieve similar rotational positions as the connector 10, as discussed above and shown in FIGS. 12-17.

The following discussion relates to a method for coupling the bone screw 760 to the second connector body 418. This method may be conducted, for example, by the manufacturer or by the surgical staff. Further, this method may be conducted, for example, before the connector 410 is positioned within the body (e.g., by the manufacturer or the surgical staff) or after the bone screw 760 is coupled to the vertebral member by surgical staff. Additionally, in this method, the threaded fastener 420 is already partially inserted into the second connector body 418. In a first operational step, the retainer 750 is placed above the head 940 of the bone screw 760, and the head 940 is then moved upwardly (e.g., in the direction of the second end 604 of the second connector body 418) and received within the aperture 632 adjacent the threaded interface 638. As the head 940 is moved upwardly into the interior of the second connector body 418, the head 940 presses the retainer 750 upwardly into the second groove 820 (if the retainer 750 is not already located within the second groove 820). As the head 940 continues to move upwardly in the direction of the threaded interface 638, the first surface 904 of the retainer 750 abuts against the continuous annular surface 850 stopping upward movement of the retainer 750 and forcing outward movement of the retainer 750 towards the cylindrical surface 854 of the second groove 820 as the head 940 continues in an upward direction. The retainer 750 begins to contract about the outer spherical surface 946 as the center of the head 940 passes beyond the center of the second groove 820. The retainer 750 is then moved down into a final operative position in the first groove 818, shown in FIG. 22, by either an upward pull on the second connector body 418 or, in some cases, by driving the bone screw 760 (via the inner drive 944) further into the vertebral member. If the threaded fastener 420 is not already inserted into the second connector body 418, the threaded fastener 420 is then inserted into and mated with the threaded interface 638 of the aperture 432 of the second connector body 418 after the bone screw 760 is inserted into the second connector body 418. With the bone screw 760 inserted into the second connector body 418, the threaded fastener 420 is rotated, using a surgical tool engaged with the inner drive 972, until a selected pressure is reached at which point the inner surface 978 of the threaded fastener 420 is biased against the outer spherical surface 946. Also, as the threaded fastener 420 rotates and moves downwardly into the second connector body 418, the threaded fastener 420 presses downwardly against the head 940, which urges the bone screw 760 toward the retainer 750 and into locking engagement therewith. The retainer 750 thus frictionally abuts the outer spherical surface 946 and expands outwardly against the cylindrical surface 834 of the first groove 818. In some examples, about 80 to about 120 inch pounds of torque on the threaded fastener 420 may be applied for fixing the bone screw 760 with respect to the second connector body 418.

In some aspects, the connector 410 can be assembled using the operational steps discussed above with respect to FIG. 18. In some aspects, the second connector body 418 may be coupled to the first connector body 414, and then the connector 410 may be coupled to the bone screw 760, as discussed above. In some aspects, the second connector body 418 may be already coupled to the bone screw 760, as discussed above, before the second connector body 418 is coupled to the first connector body 414.

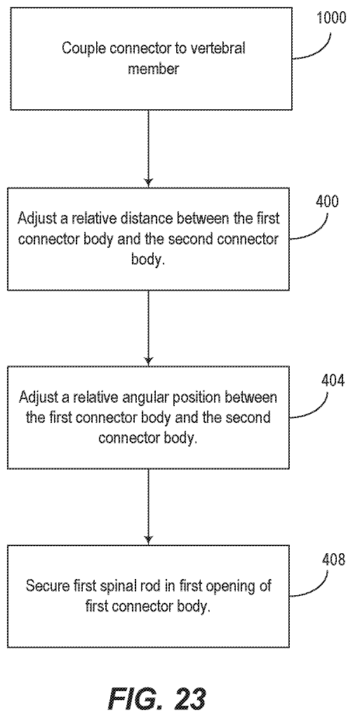

As noted above, the connector 410 is configured to receive and secure the first spinal rod S1 to the bone screw 760. Referring to FIG. 23, an exemplary method of use of the present connector 410 is described. FIG. 23 is a modified exemplary method that is similar to that shown and discussed relative to FIG. 19, so only the differences will be discussed. For example, at operational step 1000, the connector 410 may be coupled to the vertebral member. That is, in some aspects, as discussed above, the connector 410, with the bone screw 760 already coupled thereto, may be coupled to the vertebral member. Alternatively, in other aspects, the bone screw 760 may be coupled to the vertebral member and then the connector 410, via the second connector body 418, may be coupled to the bone screw 760. Once the connector 410 is appropriately secured to the vertebral member and once the first spinal rod S1 is also secured to the vertebral members, at operational steps 400 and 404, the user may adjust the relative distance and angular position of the first and second connector bodies 414, 418 to the relative positions of the first spinal rod S1 and bone screw 760, as discussed above. At operational step 408, the user may secure the first spinal rod S1 within the first opening 454 to achieve the permanently coupled state, as discussed above.

The term “substantially” as used herein may be applied to modify any quantitative representation which could permissibly vary without resulting in a change in the basic function to which it is related. For example, the connector 10 as disclosed herein having a first connector body 14 and a second connector body 18 may permissibly have plus or minus two degrees of rotation within the scope of the invention, if its ability to connect two spinal rods is not materially altered.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the invention should not be limited by any of the above-described exemplary embodiments, but should be defined only in accordance with the claims and their equivalents.

The techniques of this disclosure may also be described in the following examples.

Example 1: A connector for use in a spinal procedure, the connector comprising:
a first connector body including a first opening extending therethrough along a first axis and a support recess extending therethrough along a second axis that is transverse to the first axis;
a second connector body including a second opening extending therethrough and a post extending laterally therefrom, the post supported by the support recess and having a groove;
a fastener secured within the first connector body and having an end that is positioned within the support recess and the groove; and
a crown supported by the first connector body and positioned within the first opening, the crown including a first crown recess that engages the post and a second crown recess that is opposite the first crown recess,
wherein the second connector body is translatable and rotatable relative to the first connector body to adjust a position of the first opening relative to the second opening, and
wherein surfaces of the support recess and the first crown recess create an interference fit against the post to temporarily maintain the position of the first opening relative to the second opening.

Example 2: The connector of Example 1, wherein the second crown recess is configured to support a first spinal rod within the first opening and the second opening is configured to receive a second spinal rod.

Example 3: The connector of Example 1, wherein the groove includes a first end and a second end opposite the first end, and wherein the second connector body is movable between a first axial position and a second axial position, in the first axial position the end of the fastener is positioned against the second end of the groove and the second connector body is spaced apart from the first connector body, in the second axial position the end of the fastener is positioned against the first end of the groove and the second connector body abuts the first connector body.

Example 4: The connector of Example 1, wherein the second connector body is rotatable relative to the first connector body along a 60-degree arc.

Example 5: The connector of Example 1, wherein the groove includes a first end, a second end opposite the first end, and a recessed surface extending between the first end and the second end, and wherein the recessed surface engages the end of the fastener to prevent the second connector body from rotating beyond a predetermined angle relative to the first connector body.

Example 6: The connector of Example 1, wherein the first axis and the second axis define a plane and the second connector body defines a third axis, and wherein the second connector body is movable about the second axis from a first rotational position in which the third axis is within the plane to a second rotational position in which the third axis is oriented at a predetermined angle relative to the plane.

Example 7: The connector of Example 6, wherein the groove includes a first end, a second end opposite the first end, and a recessed surface extending between the first end and the second end, and wherein the end of the fastener is positioned against the recessed surface of the groove in the second rotational position to prevent the second connector body from rotation beyond the second rotational position.

Example 8: A rod-to-rod connector for use in a spinal procedure, the rod-to-rod connector comprising:
a first connector body including a first opening extending therethrough along a first axis and a support recess extending therethrough along a second axis that is transverse to the first axis;
a second connector body including a second opening extending therethrough and a post extending laterally therefrom, the post supported by the support recess and having a groove;
a pin secured within the first connector body and having a first end and a second end opposite the first end, the second end being positioned within the support recess and the groove; and
a crown supported by the first connector body and positioned within the first opening, the crown including a first crown recess that engages the post and a second crown recess that is opposite the first crown recess,
wherein the second connector body is movable along the second axis to allow adjustment of a distance between the first connector body and the second connector body, and
wherein the second connector body is movable about the second axis to allow adjustment of an angular position between the first connector body and the second connector body.

Example 9: The rod-to-rod connector of Example 8, wherein surfaces of the support recess and the first crown recess create an interference fit against the post to temporarily maintain the distance and angular position between the first connector body and the second connector body.

Example 10: The rod-to-rod connector of Example 8, wherein the second crown recess is configured to support a first spinal rod within the first opening and the second opening is configured to receive a second spinal rod.

Example 11: The rod-to-rod connector of Example 10, wherein the first opening defines a first threaded interface that is configured to matingly receive a threaded fastener, the threaded fastener configured to exert a force on the first spinal rod such that the support recess and the first crown recess create a taper lock against the post to prevent the second connector body from moving relative to the first connector body.

Example 12: The rod-to-rod connector of Example 8, wherein the groove includes a first end and a second end opposite the first end, and wherein the second connector body is movable between a first axial position and a second axial position, in the first axial position the second end of the pin is positioned against the second end of the groove and the second connector body is spaced apart from the first connector body, in the second axial position the second end of the pin is positioned against the first end of the groove and the second connector body abuts the first connector body.

Example 13: The rod-to-rod connector of Example 8, wherein the second connector body is rotatable relative to the first connector body along a 60-degree arc.

Example 14: The rod-to-rod connector of claim 8, wherein the groove includes a first end, a second end opposite the first end and a recessed surface extending between the first end and the second end, and wherein the recessed surface engages the second end of the pin to prevent the second connector body from rotating beyond a predetermined angle in a first direction and a second direction opposite the first direction.

Example 15: The rod-to-rod connector of Example 8, wherein the first axis and the second axis define a plane and the second connector body defines a third axis, and wherein the second connector body is movable about the second axis from a first rotational position in which the third axis is within the plane to a second rotational position in which the third axis is oriented at a first predetermined angle relative to the plane.

Example 16: The rod-to-rod connector of Example 15, wherein the groove includes a first end, a second end opposite the first end, and a recessed surface extending between the first end and the second end, and wherein the second end of the pin is positioned against the recessed surface of the groove in the second rotational position to prevent the second connector body from rotation beyond the second rotational position.

Example 17: A method of assembling a rod-to-rod connector for use in a spinal procedure, the connector including a first connector body, a second connector body, a crown, and a fastener, the first connector body having a first axis and a support recess extending along a second axis, the second connector body having a post extending laterally therefrom, the post defining a groove, the crown positioned within the first connector body and defining a first crown recess, the method comprising:
positioning the crown within the first connector body such that the first crown recess faces the support recess;
positioning the post of the second connector body between the support recess and the first crown recess such that the groove is positioned within the support recess; and securing the fastener within the first connector body such that an end of the fastener extends into the recess of the first connector body and the groove of the post, the second connector body being translatable and rotatable relative to the first connector body.

Example 18: The method of Example 17, further comprising creating, via engagement between the post and surfaces of each of the support recess and the first crown recess, an interference fit on the post to temporarily maintain the position of the first connector body relative to the second connector body.

Example 19: The method of Example 17, wherein securing the fastener within the first connector body includes welding the fastener within the first connector body.

Example 20: The method of Example 17, wherein positioning of the crown within the first connector body such that the support recess faces the first crown recess includes advancing the crown into the first connector body along the first axis with a longitudinal axis of the first crown recess oriented transverse to the second axis and rotating the crown about the first axis to position the longitudinal axis of the first crown recess parallel to the second axis.

Example 21: A connector for use in a spinal procedure, the connector comprising:
a first connector body including a first opening extending therethrough along a first axis and a support recess extending therethrough along a second axis that is transverse to the first axis;
a second connector body including a post extending laterally therefrom, the post supported by the support recess and having a groove;
a first fastener secured within the first connector body and having an end that is positioned within the support recess and the groove;
a second fastener secured to and extending from the second connector body, the second fastener configured to couple to a vertebral member; and
a crown supported by the first connector body and positioned within the first opening, the crown including a first crown recess that engages the post and a second crown recess that is opposite the first crown recess,
wherein the second connector body is translatable and rotatable relative to the first connector body to adjust a position of the first opening relative to the second fastener.

Example 22: The connector of Example 21, wherein the second crown recess is configured to support a first spinal rod within the first opening.

Example 23: The connector of Example 21, wherein the groove includes a first end and a second end opposite the first end, and wherein the first connector body is movable between a first axial position and a second axial position, in the first axial position the end of the first fastener is positioned against the second end of the groove and the second connector body is spaced apart from the first connector body, in the second axial position the end of the first fastener is positioned against the first end of the groove and the second connector body abuts the first connector body.

Example 24: The connector of Example 21, wherein the second connector body is rotatable relative to the first connector body along a 60-degree arc.

Example 25: The connector of Example 21, wherein the groove includes a first end, a second end opposite the first end, and a recessed surface extending between the first end and the second end, and wherein the recessed surface engages the end of the first fastener to prevent the second connector body from rotating beyond a predetermined angle relative to the first connector body.

Example 26: The connector of Example 21, wherein the first axis and the second axis define a plane and the second connector body defines a third axis, and wherein the first connector body is movable about the second axis from a first rotational position in which the third axis is within the plane to a second rotational position in which the third axis is oriented at a predetermined angle relative to the plane.

Example 27: The connector of Example 26, wherein the groove includes a first end, a second end opposite the first end, and a recessed surface extending between the first end and the second end, and wherein the end of the first fastener is positioned against the recessed surface of the groove in the second rotational position to prevent the first connector body from rotation beyond the second rotational position.

Example 28: The connector of Example 21, wherein surfaces of the support recess and the first crown recess create an interference fit against the post to temporarily maintain the position of the first opening relative to the second fastener.

Various examples of the disclosure have been described. These and other examples are within the scope of the following claims.

1-15. (canceled)

16. A connector for use in a spinal procedure, the connector comprising:
a first connector body including a first opening extending therethrough along a first axis and a support recess extending therethrough along a second axis that is transverse to the first axis;
a second connector body including a second opening extending therethrough and a post extending laterally therefrom, the post supported by the support recess and having a groove;
a fastener secured within the first connector body and having an end that is positioned within the support recess and the groove; and
a crown supported by the first connector body and positioned within the first opening, the crown including a first crown recess that engages the post and a second crown recess that is opposite the first crown recess,
wherein the second connector body is translatable and rotatable relative to the first connector body to adjust a position of the first opening relative to the second opening, and
wherein surfaces of the support recess and the first crown recess create an interference fit against the post to temporarily maintain the position of the first opening relative to the second opening.

17. The connector of claim 16, wherein the second crown recess is configured to support a first spinal rod within the first opening and the second opening is configured to receive a second spinal rod.

18. The connector of claim 16, wherein the groove includes a first end and a second end opposite the first end, and wherein the second connector body is movable between a first axial position and a second axial position, in the first axial position the end of the fastener is positioned against the second end of the groove and the second connector body is spaced apart from the first connector body, in the second axial position the end of the fastener is positioned against the first end of the groove and the second connector body abuts the first connector body.

19. The connector of claim 16, wherein the second connector body is rotatable relative to the first connector body along a 60-degree arc.

20. The connector of claim 16, wherein the groove includes a first end, a second end opposite the first end, and a recessed surface extending between the first end and the second end, and wherein the recessed surface engages the end of the fastener to prevent the second connector body from rotating beyond a predetermined angle relative to the first connector body.

21. The connector of claim 16, wherein the first axis and the second axis define a plane and the second connector body defines a third axis, and wherein the second connector body is movable about the second axis from a first rotational position in which the third axis is within the plane to a second rotational position in which the third axis is oriented at a predetermined angle relative to the plane.

22. The connector of claim 21, wherein the groove includes a first end, a second end opposite the first end, and a recessed surface extending between the first end and the second end, and wherein the end of the fastener is positioned against the recessed surface of the groove in the second rotational position to prevent the second connector body from rotation beyond the second rotational position.

23. A rod-to-rod connector for use in a spinal procedure, the rod-to-rod connector comprising:
a first connector body including a first opening extending therethrough along a first axis and a support recess extending therethrough along a second axis that is transverse to the first axis;
a second connector body including a second opening extending therethrough and a post extending laterally therefrom, the post supported by the support recess and having a groove;
a pin secured within the first connector body and having a first end and a second end opposite the first end, the second end being positioned within the support recess and the groove; and
a crown supported by the first connector body and positioned within the first opening, the crown including a first crown recess that engages the post and a second crown recess that is opposite the first crown recess,
wherein the second connector body is movable along the second axis to allow adjustment of a distance between the first connector body and the second connector body, and
wherein the second connector body is movable about the second axis to allow adjustment of an angular position between the first connector body and the second connector body.

24. The rod-to-rod connector of claim 23, wherein surfaces of the support recess and the first crown recess create an interference fit against the post to temporarily maintain the distance and angular position between the first connector body and the second connector body.

25. The rod-to-rod connector of claim 23, wherein the second crown recess is configured to support a first spinal rod within the first opening and the second opening is configured to receive a second spinal rod.

26. The rod-to-rod connector of claim 25, wherein the first opening defines a first threaded interface that is configured to matingly receive a threaded fastener, the threaded fastener configured to exert a force on the first spinal rod such that the support recess and the first crown recess create a taper lock against the post to prevent the second connector body from moving relative to the first connector body.

27. The rod-to-rod connector of claim 23, wherein the groove includes a first end and a second end opposite the first end, and wherein the second connector body is movable between a first axial position and a second axial position, in the first axial position the second end of the pin is positioned against the second end of the groove and the second connector body is spaced apart from the first connector body, in the second axial position the second end of the pin is positioned against the first end of the groove and the second connector body abuts the first connector body.

28. The rod-to-rod connector of claim 23, wherein the second connector body is rotatable relative to the first connector body along a 60-degree arc.

29. The rod-to-rod connector of claim 23, wherein the groove includes a first end, a second end opposite the first end and a recessed surface extending between the first end and the second end, and wherein the recessed surface engages the second end of the pin to prevent the second connector body from rotating beyond a predetermined angle in a first direction and a second direction opposite the first direction.

30. The rod-to-rod connector of claim 23, wherein the first axis and the second axis define a plane and the second connector body defines a third axis, and wherein the second connector body is movable about the second axis from a first rotational position in which the third axis is within the plane to a second rotational position in which the third axis is oriented at a first predetermined angle relative to the plane.

31. The rod-to-rod connector of claim 30, wherein the groove includes a first end, a second end opposite the first end, and a recessed surface extending between the first end and the second end, and wherein the second end of the pin is positioned against the recessed surface of the groove in the second rotational position to prevent the second connector body from rotation beyond the second rotational position.

32. A method of assembling a rod-to-rod connector for use in a spinal procedure, the connector including a first connector body, a second connector body, a crown, and a fastener, the first connector body having a first axis and a support recess extending along a second axis, the second connector body having a post extending laterally therefrom, the post defining a groove, the crown positioned within the first connector body and defining a first crown recess, the method comprising:
positioning the crown within the first connector body such that the first crown recess faces the support recess;
positioning the post of the second connector body between the support recess and the first crown recess such that the groove is positioned within the support recess; and
securing the fastener within the first connector body such that an end of the fastener extends into the recess of the first connector body and the groove of the post, the second connector body being translatable and rotatable relative to the first connector body.

33. The method of claim 32, further comprising creating, via engagement between the post and surfaces of each of the support recess and the first crown recess, an interference fit on the post to temporarily maintain the position of the first connector body relative to the second connector body.

34. The method of claim 32, wherein securing the fastener within the first connector body includes welding the fastener within the first connector body.

35. The method of claim 32, wherein positioning of the crown within the first connector body such that the support recess faces the first crown recess includes advancing the crown into the first connector body along the first axis with a longitudinal axis of the first crown recess oriented transverse to the second axis and rotating the crown about the first axis to position the longitudinal axis of the first crown recess parallel to the second axis.
